(19) United States
(12) Patent Application Publication
(10) Pub. No.: US 2024/0180752 A1
(43) Pub. Date: Jun. 6, 2024

(54) Wound Treatment System and Wound Treatment Kit

(71) Applicant: MEDICAL HOLDING AG, Baar (CH)

(72) Inventors: Claudia Kowanda, Obrägeri (CH); Lukas Bannwart, Rotkreuz (CH)

(21) Appl. No.: 18/286,338

(22) PCT Filed: Apr. 6, 2022
(86) PCT No.: PCT/EP2022/059112

§ 371 Date: Oct. 10, 2023

(30) Foreign Application Priority Data
Apr. 14, 2021 (EP) 21168374.3

(51) Int. Cl. A61F 13/02; A61M 1/00

(52) U.S. Cl. CPC A61F 13/023; A61F 13/02; A61M 1/91

(57) ABSTRACT

A wound treatment system having a seal which can be applied to a skin surrounding the wound and a fluid-permeable pad which is arranged between the seal and a wound bed W made of a flat, essentially planar full plastic body which is penetrated by drainage passages, characterized in that at least one of the drainage passages has a valve.

The present invention relates to a wound treatment system and a wound treatment kit, in particular for wound treatment by means of negative pressure.

US 2006/0041247 A1 discloses a wound treatment kit consisting of a plastic body penetrated by a plurality of channels which penetrate the essentially flat plastic body transversely, i.e. at right angles to the main side surfaces of the plastic body, to form drainage passages so that exudate to be drawn off can pass through the plastic body. However, channels are also formed in the plane of the plastic body. These can be formed within the plastic body as closed channels or as groove-shaped channels open to the main side surfaces. In the prior art, these channels are provided with drainage passages so that the exudate to be drawn off can be collected in the channels before passing through the plastic body or afterwards in the respective channels.

In the prior art, the full plastic body is arranged above the wound and provided with a seal into which a suction hose leads. The seal is usually formed by a self-adhesive film which is adhered to the skin with the full plastic body enclosed. A negative pressure is created underneath the seal via the suction hose so that the seal is applied to the front surface of the plastic body when the exudate is drawn off.

Insofar as the exudate is transported through the plastic body via channels which are not arranged on the front surface of the plastic body, these remain free for fluid transport even when negative pressure is acting. The drainage passages ending at the front surface of the plastic body are usually relocated by the cover in the event of an acting suction pressure.

Other wound treatment systems with a fluid-permeable wound dressing are known from WO 2019/027731 A1; WO 2019/136164 A1 and WO 2008/104609 A1, respectively.

The wound treatment systems exemplarily mentioned here are usually used for the treatment of poorly healing and possibly heavily weeping wounds. The arrangement of the wound dressing underneath a cover with a suction hose is intended to make it possible to permanently drain off the exudate accumulating in the wound bed without the need to change the wound dressing, which is fundamentally not conducive to wound healing, since granulation in the wound bed and the growth of new cells is thereby disturbed.

In this context, the previously known solutions aim to ensure that exudate is reliably removed from the entire area of the wound by means of an adapted form of the wound dressing. Nevertheless, this type of permanent wound care underneath a cover poses the problem of maceration. This can be avoided on healthy skin by using a breathable cover. However, the problem of exudate not being completely drained from the area underneath the wound dressing and the associated risk of maceration has not yet been fully solved.

The present invention is based on the problem of providing a wound treatment system of the type mentioned introductorily which addresses the problem of maceration in an improved manner and provides for better removal of exudate.

In order to solve this problem, the present invention proposes a wound treatment system with the features of claim 1.

This wound treatment system has, in a manner known, a seal which is adhered to the skin surrounding the wound. The seal is preferably a self-adhesive, breathable film, for example an incision film.

The wound treatment system according to the present invention has a full plastic body which is planar and substantially flat.

Preferably, the full plastic body is made of a biocompatible plastic, in particular silicone. The plastic material can be formed with or without porosity, i.e. as a foam. The full plastic body is preferably manufactured by injection molding or extrusion. It is also conceivable to manufacture it by means of 3-D printing or casting.

As a preferably completely flat body, the full plastic body has a flat surface on its rear side facing the wound bed and also a flat surface on its top side facing away from the wound bed. The flat surfaces on the front and rear sides are usually perforated by structures. Thus, an entrance opening of at least one drainage passage is formed on the lower side. The drainage passage can end at a rear lower surface of the full plastic body, i.e. open into it, or have an opening which is remote from the lower surface and is located, for example, in a channel formed on the underside extending transversely to the full plastic body or in a recess surrounding the opening. In particular, however, on the upper side of the full plastic body, which is to be arranged facing away from the wound, various structures for guiding the flow are provided, which will be discussed below.

The full plastic body has a valve to at least one drainage passage, preferably to at least 50% of the drainage passages, and particularly preferably to all drainage passages. This valve allows the fluid to pass through to the upper side facing away from the wound when suction pressure is applied, but impedes or prevents backflow in the absence of a pressure gradient from the wound bed in the direction of the upper side of the full plastic body.

The valve is usually molded in one piece on the full plastic body. The valve is regularly produced from a still molten plastic during the primary molding of the full plastic body. The valve is thus preferably an integral part of the full plastic body.

Various configurations are conceivable with which an opening and closing movement of the valve is also possible. For example, a non-return valve can be provided inside the full plastic body, which opens due to the acting suction pressure and, due to the elasticity of the plastic material, springs back via the valve body into a position in which the valve is closed when there is no pressure difference. Such a non-return valve may be formed by a valve flap extending substantially transversely of the drainage passage and connected at one side to the remainder of the full plastic body, but not connected thereto at the opposite side. Such a valve can be completely formed in the aforementioned manner by master forming. However, such a valve flap can also be formed by post-processing, for example by punching, slitting or cutting free an initially continuously formed diaphragm.

In this case, the valve is usually designed in such a way that, at least in the open state, the valve is lower than the front surface formed at the front by the full plastic body. This applies, for example, if the valve forms a valve opening in extension of the drainage passage. This valve opening is below the front surface when fluid passes through the drainage passage. In this case, the valve may be provided at the end of the drainage passage associated with the upper surface. However, the valve can also be located within the drainage passage, if necessary also adjacent to the lower surface.

In order to ensure good discharge of the drained exudate, the valve opening is surrounded by a valve channel, the base of which is lower than the valve opening, i.e. further away from the front side of the full plastic body. In this way, fluid discharged from the drainage passage or the valve can reach a lower level, which in itself prevents the exhausted fluid from re-entering the drainage passage. In such a configuration, it is not necessary for the valve to form movable flaps. Rather, the valve function is effected by preventing backflow back into the drainage passage. In this context, it is not necessary, but advantageous, for the full plastic body to lie lower in the earth's gravitational field with its lower surface than with its surface after the wound has been dressed. This is because the full plastic body is usually under suction pressure, which generates a potential that causes the fluid on the front side to be conveyed away from the respective drainage passages. In the previously discussed configuration, the valve channel is located lower than the valve opening, i.e. further from the surface of the full plastic body than the valve opening. In order to prevent displacement of the valve opening, the valve channel is preferably surrounded by a collar extending from the surface. The cover spanning the surface or a separate film, which will be discussed below, accordingly projects beyond the valve opening at a distance so that the valve opening remains open at all times and even in the event of an acting negative pressure.

In addition to this valve function by preventing backflow due to the formation of the valve opening on a higher potential level than the walls surrounding the valve opening, the valve can also be cone-shaped with a closing valve slot that allows fluid to pass through only when a negative pressure is acting.

Alternatively, a valve can be designed to open laterally, i.e. transversely to the main side surfaces of the full plastic body formed by the top surface and the bottom surface. For this purpose, the valve has exposed valve lugs towards a drainage channel. These valve lugs form between them an outlet which opens laterally, are usually provided at the edge of the drainage channel, and are particularly preferably recessed in the channel wall. Also in this configuration, the drainage channel is usually lower than the outlet opening between the valve lugs so that fluid discharged through the valve lugs does not stand at the outlet opening, but is discharged at a lower level.

According to an alternative configuration, the valve comprises valve lugs which delimit a drainage passage at its upper side and usually cover it. These valve lugs are exposed in a valve channel which can receive the fluid emerging from the valve, but in any case is dimensioned in such a way that the valve lugs can move within limits in the valve channel, in particular can be compressed.

A film is applied to the top of the full plastic body, usually to its surface, in this variant. This film interacts with the valve lugs underneath the film and inside the full plastic body when negative pressure is applied. This is because the film is deformed downwards, i.e. in the direction of the wound bed, when negative pressure is applied. It then lies against the valve lugs, which are spaced further apart from the surface than the film. The deformed film causes deformation of the valve lugs, which are consequently spread apart. The spreading causes opposing inner surfaces of the valve lugs, which are provided in extension of the drainage passage, to be spaced apart so that the passage through the valve is free. The valve lugs can be connected to each other at the upper side or closed by the film. The fluid exits laterally, for example, through a transverse valve channel formed between the valve lugs. Thus, the valve lugs are usually located directly adjacent to the drainage channel. In this context, the valve lugs can form a valve opening towards the drainage channel and be closed on the opposite side, for example by contact with the remaining full plastic body. However, the valve lugs can also be substantially completely surrounded by the valve channel.

The aforementioned lateral opening of the drainage passages into an associated drainage channel can also form a valve by itself and without a valve lug. This variant assumes that preferably the drainage passages communicate with drainage channels which are open towards the front side. The drainage channels are accordingly formed on the front side of the full plastic body facing away from the wound bed.

The drainage channels are usually fluid-tight to the opposite rear side. In other words, a bottom of the drainage channel does not communicate directly with a drainage passage, or the bottom does not have such a drainage passage. Rather, the outlet opening of the drainage passage is located above the bottom of the drainage channel, which already creates a potential difference and thus a valve effect. In any case, the fluid collected in the channel cannot pass directly through the full plastic body in the direction of the wound bed.

In the variant discussed here, the outlet opening of the drainage passage is located in a channel wall delimiting the drainage channel. Here, too, the drainage passage opens laterally into the drainage channel. The outlet opening is located below the surface and above the bottom of the drainage channel. In the thickness direction of the full plastic body and starting from the rear side, the bottom of the drainage channel is then located lowest, followed above by the outlet opening at the edge of the drainage channel, which in turn is located below the surface of the full plastic body, but possibly ends in the plane containing the surface.

As can be seen from the above description, the full plastic body is covered with a film on its front side. This film is a cover or seal different from the seal according to claim 1. This film is usually already applied, glued, to the upper side of the full plastic body from the outset. Thus, the full plastic body together with the film applied thereto is delivered to the user as part of a wound treatment kit in a usually sterile package. In other words, the full plastic body is already sealed with the film on its front side from the outset. Thus, the full plastic body together with the film basically forms sealed capillaries on its front side, which communicate with the wound bed on the rear side only via the drainage passages. On the front side, there is usually only a suction hose connection which communicates with the flow channels inside the full plastic body. This suction hose connection projects beyond the otherwise flat surface of the full plastic body. The suction hose connection forms interfaces for the direct connection of a suction hose. The suction hose connection can be designed in the form of a suction hose connection web for the indirect or direct connection of a hose. The hose connection web can be part of this suction hose connection web and can cooperate, for example, with a connection cap that is attached to the hose connection web and communicates with the drainage channels and/or the drainage passages. The connection of a hose can be provided by a separate suction hose connection element, which is connected to the full plastic body after the film has been applied to it. The suction hose connection element can also provide an interface for the connection of a rinsing hose and accordingly also be a rinsing hose connection. Accordingly, the hose connection element can provide both the connection of a suction hose and a rinsing hose and, for this purpose, can form at least one mechanical interface in one piece on the full plastic body.

For a secure seal, a sealing surface is preferably provided adjacent to the suction hose connection. This sealing surface is characterized by the fact that it has a defined, essentially closed circumference within the plane of the full plastic body, which is penetrated only within the plane of the full plastic body by a discharge channel for the drained fluid and/or a rinsing supply channel for a rinsing fluid to be supplied. The rinsing fluid is preferably controlled air introduced under the film through a rinsing valve.

Alternatively, a hose can also be guided under the seal to the full plastic body and laid over, under or next to the full plastic body and/or open out there.

The sealing surface is usually located in the one flat surface of the full plastic body. The suction hose connection element has at least one, preferably two, connectors for hoses for suction or rinsing, which project from a flat fastening base of the suction hose connection element. This fastening base is connected to the full plastic body during wound care, for example via the seal cut out in the area of the connectors. The fastening base rests against the sealing surface so that the discharge channel or the rinsing supply channel communicate in a sealed manner with the respective connectors and thus the hoses connected to them. During wound care, the seal is placed over the full plastic body and cut into in the area of the connectors so that they project beyond the seal, but the seal is otherwise placed on or glued to the surface of the fastening base.

The aforementioned configuration of a full plastic body, which both forms part of the wound dressing and also integrally forms the suction hose connection or provides an interface thereto, can per se be essential to the invention. The further development does not necessarily require a valve associated with the drainage passage.

Apart from the openings of the drainage passages, the full plastic body preferably forms a flat contact surface on its rear side associated with the wound bed. This can be applied tightly against the areas of the skin bordering the wound bed. This measure prevents the wound fluid from spilling over onto the healthy skin under the wound margin, which is sealed. A marginal area to be applied to the skin can be free of drainage passages. Thus, a marginal area of the full plastic body is adapted to seal the margins of the wound. However, this area should be configured to be breathable in order to allow the skin to breathe during prolonged wound treatment with the full plastic body.

For the aforementioned rinsing, the full plastic body preferably has a raised edge running around in the circumferential direction, which delimits a rinsing channel. The rinsing channel ex-tends in the circumferential direction, i.e. surrounds the drainage channels on the outside circumference and communicates with them. The rinsing channel communicates with the previously mentioned connector via the rinsing supply channel, which is an example of a rinsing hose connection.

Thus, different lines can be connected to the supplied wound and the full plastic body via the suction hose connection and the rinsing hose connection. Draining can be carried out with sterile fluid, for example saline solution or therapeutic rinsing fluids, in order to treat the wound bed with medication over a certain period of time or merely to effect improved drainage of exudate when the rinsing line is open.

For easier attachment of the film, an adhesive layer is provided between the full plastic body and the film. This can be applied to the film during assembly. Preferably, however, the adhesive layer is applied to the surface of the full plastic body and only there. Thus, there is no risk of the film sticking to deeper-lying areas of the full plastic body that are assigned to the transport of the fluid. Such adhesion is to be feared in particular if the film is deformed in the direction of the full plastic body when negative pressure is applied.

Alternatively, the film can also be welded to the full plastic body. In principle, any connection between the film and the full plastic body, in particular a material bond, is conceivable.

The full plastic body can also be provided with an adhesive layer on its rear side facing the wound bed. This adhesive layer is usually initially protected by a peel-off film. It is only removed by the user of the wound treatment kit. The adhesive layer may be provided over all or part of the rear side. It can serve to fix the full plastic body to the wound or to connect the full plastic body to a further wound dressing to be determined by the user, for example made of a foam or a porous silicone. The adhesive layer can also serve to fix the full plastic body to the wound, for which purpose the full plastic body can be bonded to the healthy skin surrounding the wound via the adhesive layer. For such a case, it may be sufficient if only the edge region of the full plastic body is provided with an adhesive layer on the rear side. Silicone- or acrylic-based adhesives can be used for this purpose.

In this context, it should be noted that the full plastic body can only be inadequately adapted to deep wounds or wounds with considerable topography due to its flat configuration. In such cases, the full plastic body can be combined with other fluid-permeable wound dressings known, which can be placed between the full plastic body and the wound bed.

According to a preferred further development of the present invention, the full plastic body is adapted to deliver an instill fluid in the direction of the wound bed. For this purpose, an instill outlet is preferably provided on the rear side of the full plastic body facing the wound bed. This instill outlet passes through the full plastic body in the thickness direction. The instill outlet is preferably to be arranged essentially centrally above the wound and enables a two-dimensional distribution within the wound, especially in conjunction with an additional wound dressing which is permeable to fluids and can be formed, for example, from an open-pored foam. Thus, the instill outlet is usually located in a central area of the full plastic body.

With regard to an easily attachable connection of such an instill outlet, the full plastic body preferably has an instill connection for an instill hose, via which the instill fluid for instillation is supplied to the wound. This instill fluid can be extracted from the wound bed by applying a negative pressure. Thus, in conjunction with the negative pressure, the instill fluid can be cyclically supplied to the wound and then removed together with exudate via a suction pump. However, the instill fluid can also remain in the wound if the plastic body is used without drawing off the exudate.

For uniform distribution of the instill fluid, the instill outlet preferably communicates with instill channels or is provided in the form of instill channels. The instill channels are formed on the rear side of the full plastic body as channels closed towards the front. These instill channels preferably communicate regularly with the instill connection via the instill outlet.

The flow paths for the instill fluid formed on the full plastic body are usually provided fluidi-cally separate from the drainage passages and channels or the flow paths for rinsing. While the rinsing channels usually merge into the drainage channels, the flow paths for the instill fluid are formed separately. There is no direct fluidic connection via flow paths formed on or in the full plastic body between the rinsing or drainage channels and the instill channels or the instill outlet.

The instill connection can be formed by the hose connection, which can connect at least two, preferably three lumens. In the case of two lumens, these are assigned to suction and rinsing. The third lumen is used to supply the instill fluid. Thus, the hose connection element preferably has interfaces for three hoses with the previously described functions. Further details and ad-vantages of the present invention will be apparent from the following description in conjunction with the drawing. Therein:

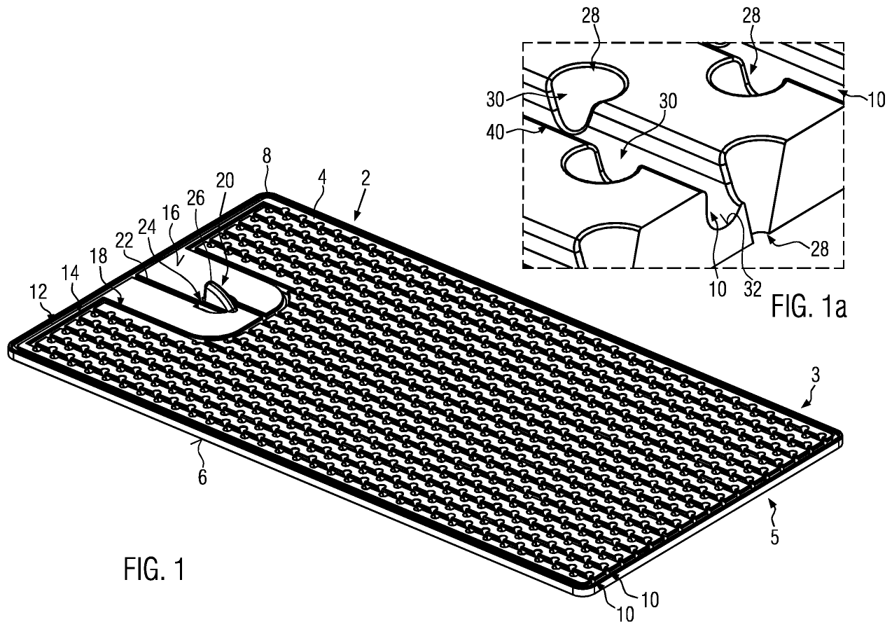

FIG. 1 shows a perspective top view of a first embodiment of a full plastic body;

FIG. 1a shows an enlarged detail of the embodiment according to FIG. 1;

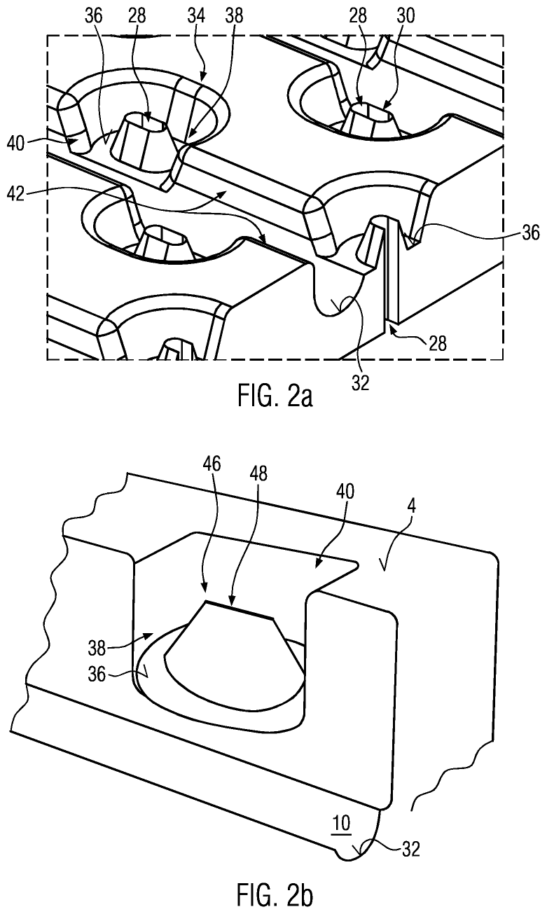

FIG. 2a shows a perspective top view of an enlarged detail of a second embodiment of a full plastic body;

FIG. 2b shows a perspective top view of an enlarged detail of a variant of the second embodiment of a full plastic body;

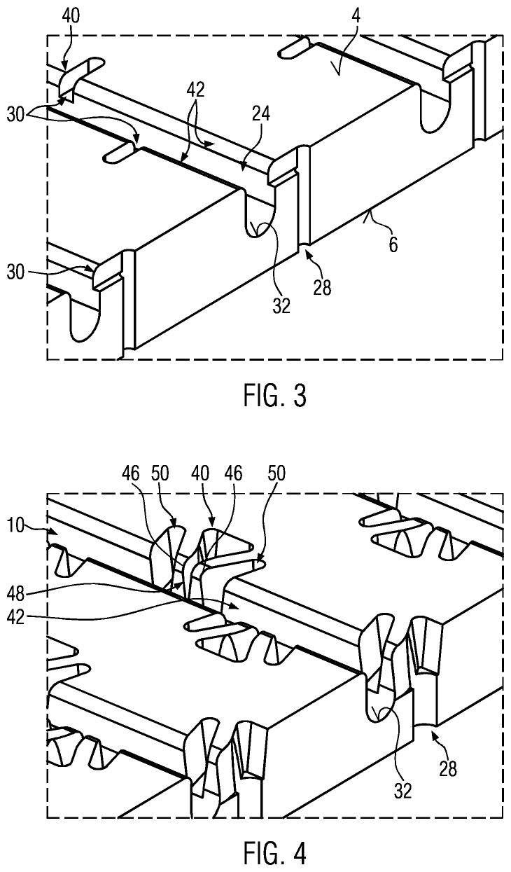

FIG. 3 shows a perspective top view of an enlarged detail of a third embodiment of a full plastic body;

FIG. 4 shows a perspective top view of an enlarged detail of a fourth embodiment of a full plastic body;

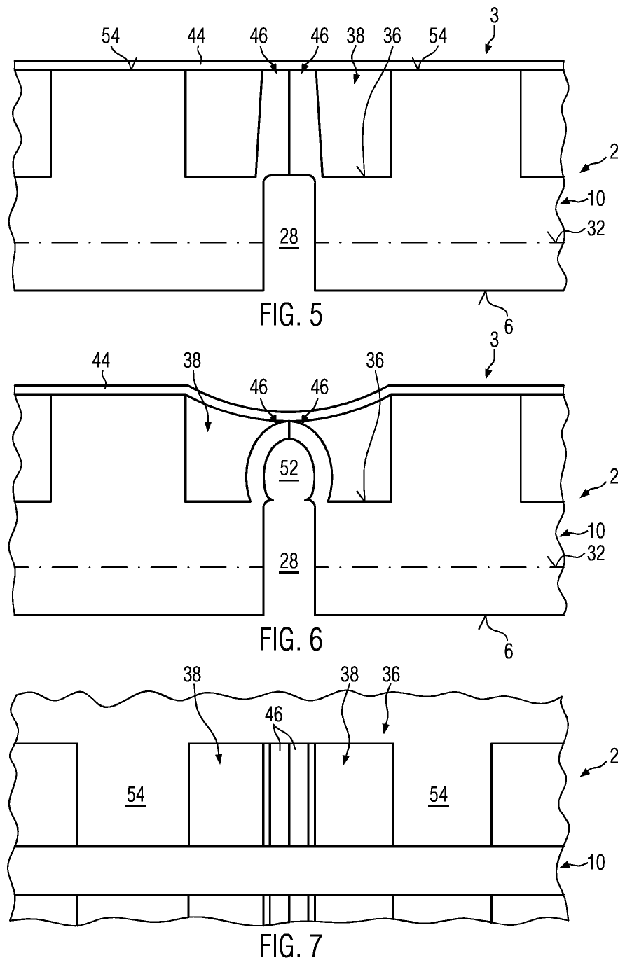

FIG. 5 shows a cross-sectional view of a fifth embodiment of a full plastic body with a film applied thereto:

FIG. 6 shows the embodiment shown in FIG. 5 during suction;

FIG. 7 shows a top view of the embodiment according to FIGS. 5 and 6;

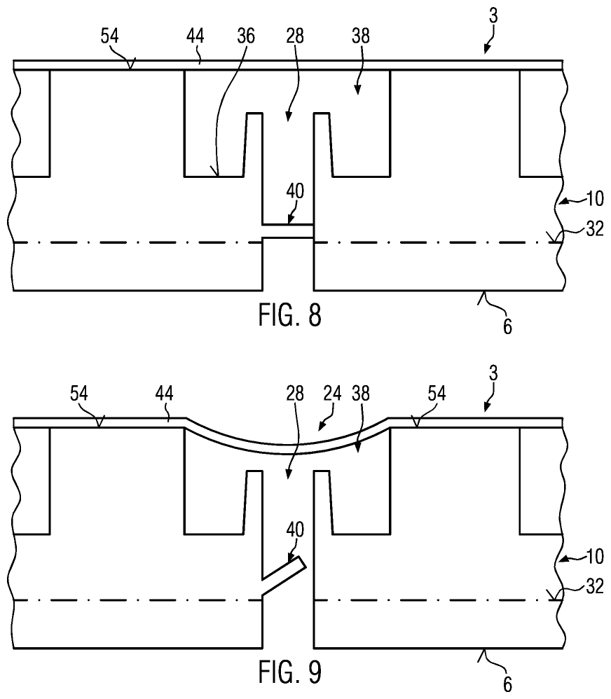

FIG. 8 shows a cross-sectional view of a sixth embodiment of a full plastic body with a film applied thereto:

FIG. 9 shows the illustration according to FIG. 8 during suction;

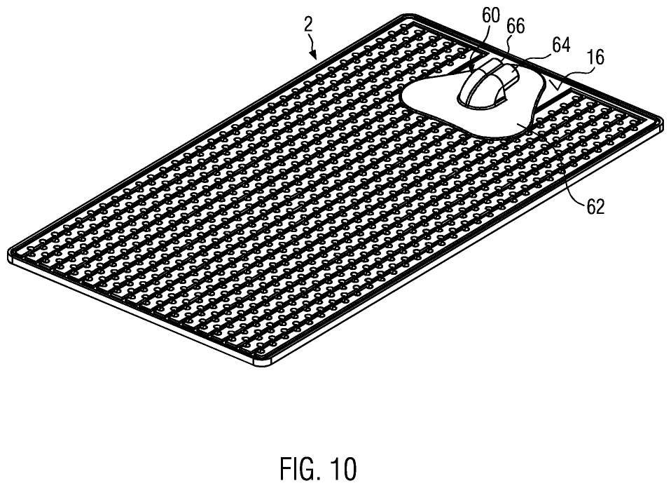

FIG. 10 shows a perspective top view of the embodiment of a full plastic body according to FIG. 2a with a suction hose connection element;

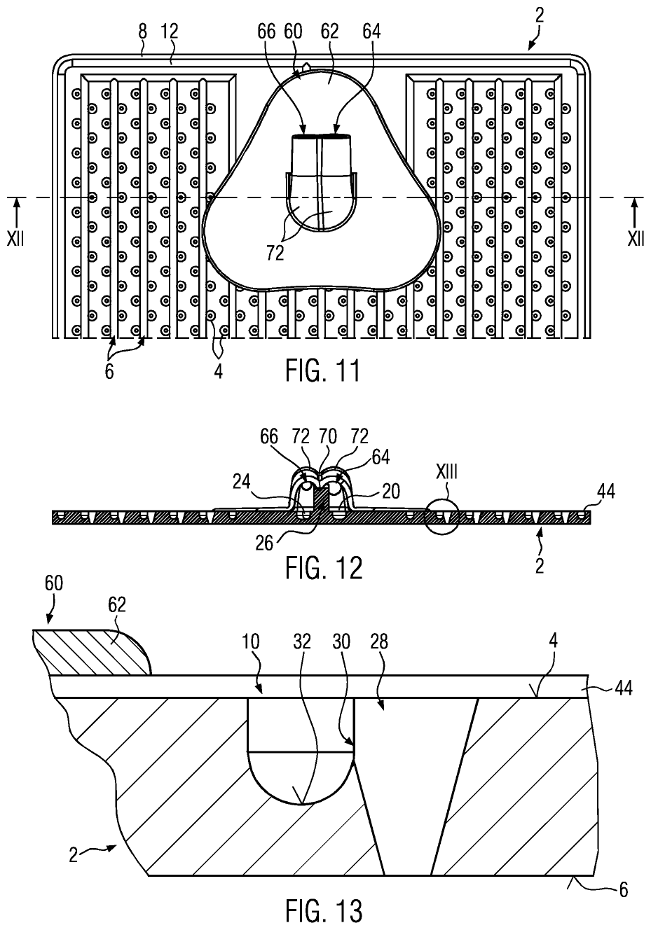

FIG. 11 shows a top view of a part of the embodiment according to FIG. 10;

FIG. 12 shows a sectional view along lines XII-XII according to FIG. 11;

FIG. 13 shows a sectional view of a detail XIII according to FIG. 12 in enlarged view;

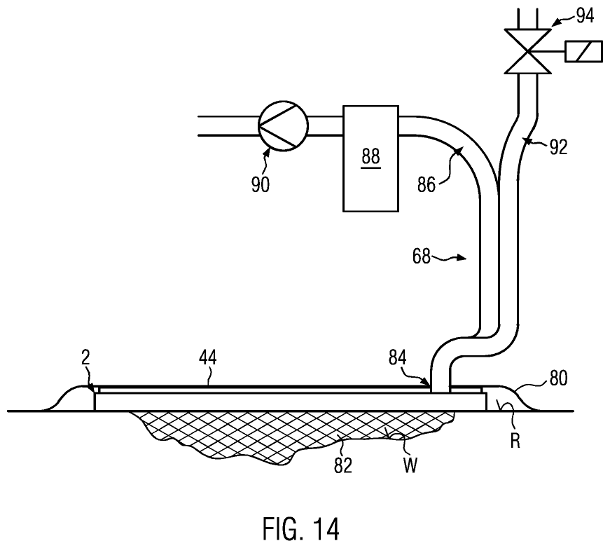

FIG. 14 shows a schematic representation of an embodiment of a wound treatment system after treatment of a wound;

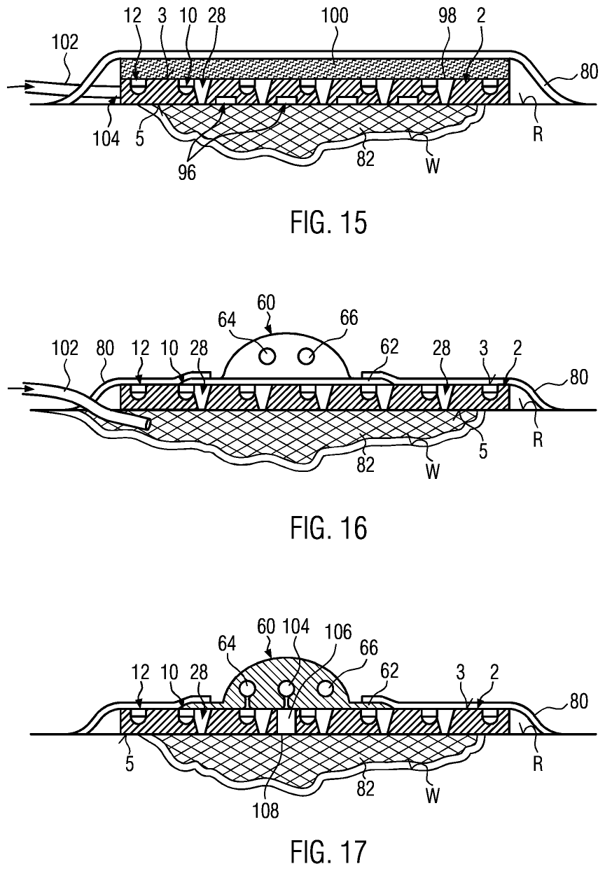

FIG. 15 shows a sectional view of a seventh embodiment of a full plastic body based on the first embodiment;

FIG. 16 shows a sectional view of the first embodiment during wound care with instillation; and

FIG. 17 shows a sectional view according to FIGS. 15 and 16 of an eighth embodiment of a full plastic body.

The Figures each show a full plastic body, characterized by reference sign 2, which forms a flat upper surface 4 on its front side 3 facing the visible side according to FIG. 1 and a likewise flat lower surface 6 on a rear side 5. The surfaces 4, 6 run parallel to each other. The full plastic body 2 is designed as a cuboid with a low height. The full plastic body 2 essentially forms a flat mat.

On the front side 3, the full plastic body 2 is provided with a circumferential raised outer edge 8, which is at the same height as the upper surface 4 and is formed to extend circumferentially.

Within this outer edge 8, a plurality of drainage channels 10 extend in the longitudinal direction of the full plastic body 2 and are open towards the front. The drainage channels 10 each run parallel to one another and, on one end face, run off from a rinsing channel 12 bounded on the outside by the outer edge 8. On the opposite side, they open into a collection channel 14 extending on the front side parallel to the rinsing channel 12. Thus, the drainage channels 10 connect in a parallel circuit the section of the rinsing channel 12 provided on the front side with the collection channel 14 provided opposite thereto. The collection channel 14 centrally surrounds a sealing surface 16, which lies in the plane of the upper surface 4, and communicates with a collecting trough 20 via a discharge channel 18.

The rinsing channel 12 communicates with a rinsing trough 24 via a rinsing supply channel 22. A raised hose connection web 26 is formed in one piece by the full plastic body 2 between the collecting trough 20 and the rinsing trough 24. This hose connection web 26 projects beyond the sealing surface 16 or the upper surface 4.

As can be seen from FIG. 1a, the full plastic body 2 is penetrated by a plurality of drainage passages 28. These drainage passages 28 extend continuously from the lower surface 6 to the upper surface 4 and accordingly penetrate the full plastic body 2 in the height direction. Below the upper surface 4, the drainage passages 28 have outlet openings 30 via which the drainage passages 28 each communicate with the associated drainage channel 10. The lower edge of these outlet openings 30, i.e. the area of the outlet opening 30 furthest from the upper surface 4, is higher and thus closer to the upper surface 4 than a bottom 32 of the drainage channel 10, thus preventing fluid that has been transferred into the drainage channel 10 from flowing back into the drainage passage 28.

A variant is shown in FIG. 2a. In the description of the Figures, similar areas and features are characterized by the same reference signs.

The embodiment according to FIG. 2a differs in the configuration and position of the drainage passage 28 from the one previously discussed. According to FIG. 2a, the drainage passage 28 ends in a chimney section which is conical on the outside and cylindrical on the inside and the outlet opening 30 of which lies below the upper surface 4 and is surrounded by a collar 34 which forms a base 36 between the plane containing the bottom 32 and the upper surface 4, which base 36 lies below the outlet opening 30 and the edge of which to the drainage channel 10 is arranged higher than the bottom 32 of the drainage channel 10 (cf. FIG. 2a). The upper side of the collar 34 lies in the surface 4.

The base 36 of the collar 34, as well as side walls of the collar 34, define a valve channel 38 that communicates with the drainage channel 10 in this manner.

The configurations shown in FIGS. 1 and 2 each prevent backflow of the fluid from the drainage channel 10 back into the drainage passage 28. This is already effected by gravity when the full plastic body 2 is aligned in the earth's gravitational field as shown in the drawing. In each of the Figures, a valve effecting this functionality is characterized by reference sign 40.

FIG. 2b shows a configuration similar to FIG. 2a. In these configurations, the valve effect of the valve 40 is enhanced by elastic deformation of wall segments formed by the full plastic body 2. For this purpose, the embodiment shown in FIG. 2b has valve lugs 46 that are elastically biased against each other to close a slot-like valve opening 48 in the initial state. A valve 40 of the non-return valve type is formed by the valve lugs 46. This valve 40 is also surrounded by a collar 34 formed in the present case with a rectangular base. Here, too, the bottom 36 of the collar 34 is higher than the bottom 32 of the drainage channel 10.

In FIG. 3, the valve 40 is formed by an outlet opening 30 recessed in a laterally upstanding channel wall 42 and communicating with the associated drainage passage 28. The drainage passages 28 are formed continuously in the height direction. The outlet openings 30 are laterally open to both the surface 4 and the drainage passage 10, and are each recessed in the opposite passage wall 42.

After applying a film 44, which is shown in FIGS. 5 to 9 and will be explained in more detail below, to the upper surface 4 of the embodiment shown in FIG. 3, only the lateral outlet opening 30 remains, which opens directly into the drainage channel 10, although here again well above the bottom 32 of the drainage channel 10. This configuration shown in FIG. 3 also prevents backflow and accordingly acts as a valve 40 in the meaning of the present invention.

The embodiment shown in FIG. 4 is formed similarly to the embodiment shown in FIG. 3. There, the outlet openings of the drainage channel 10 are realized in a channel wall 42 in the manner of a non-return valve 40. FIG. 4 shows a half-opened valve opening 48, the valve lugs 46 being provided at a distance from each other so that a direct passage from the drainage passage 28 into the drainage channel 10 is possible. To the side of the valve opening 48, longitudinal slots 50 can be seen which are recessed in the channel wall 42 and which allow the valve lugs 46 to flex elastically in response to an increased volumetric flow of the drained fluid. As before, the lower edge of the valve opening 48 is higher than the bottom 32 of the drainage channel 28.

The valve lugs 46 end flush with the surface 4 of the full plastic body 2. The configuration shown in FIG. 4 permits simple injection molding of the full plastic body 2, which has no undercuts and in which the longitudinal slots 50 or the drainage passages 28 extend strictly in the vertical direction.

The wedge-shaped configuration of the upper ends of the valve lugs 46 allows a film applied against the upper surface 4 of the full plastic body 2 to spread the valve lugs 46 when negative pressure is applied, thereby enlarging the valve opening 48. When the suction pressure decreases, the valve lugs 46 are guided towards each other again. This effect can be used to change the valve opening 48 depending on the acting pressure within the full plastic body 2. Accordingly, the valves 40 are actively opened or closed driven by the acting pressure. In other words, the valve 40 is controlled as a function of pressure and is thereby mechanically set via the film.

A corresponding principle is realized in the embodiment shown in FIGS. 5 to 7.

In this context, FIG. 7 shows the top view and illustrates that the valve lugs 46 extend up to the drainage channel 10 and—as before—are surrounded by a valve channel 38. This valve channel 38 ends above a bottom 32 of the drainage channel 10 indicated by a dashed line in FIGS. 5 and 6. Two valve lugs 46 are shown, which are separated in order to extend the drainage passage 28 between them in the open state. As a result, a valve channel 52 is formed between the valve lugs 46, which in the present case opens exclusively to the drainage channel 10 and empties into the same. The film 44 is adhered to the surface segments characterized by reference signs 54 in FIGS. 5 and 6, however not to the free ends of the valve lugs 46. This allows the valve lugs 46 to be deformed without hindrance. This configuration can be achieved by the fact that the film 44 itself has no adhesive layer, but the adhesive layer is applied exclusively to the selected surface segments 54 of the full plastic body 2.

The deformation of the valve lugs 46 results in particular from the comparison of FIGS. 5 and 6. With a negative pressure acting below the film 44 and accordingly also within the valve channel 38, the film 44 is pulled in the direction of the base 36. Since the valve lugs 46 have the same heights as the surface segments 54 in the initial state, a deformation of the valve lugs 46 results, whereby a transverse valve channel 52 is pressed free between the valve lugs 46. Drained fluid is conveyed through the drainage passage 28 into the transverse valve channel 52 and from there into the drainage channel 10. With a reduction of the suction pressure, i.e. the negative pressure between the film 44 and the full plastic body 2, there is a resetting of both the film 44 and the valve lugs 46 so that the valve lugs 46 are returned from their spread position according to FIG. 6 to their initial position according to FIG. 5 due to the elastic properties of the silicone material forming the full plastic body 2. Thus, the valve 40 is closed.

In a representation corresponding to FIGS. 5 and 6, FIGS. 8 and 9 illustrate a further embodiment in which the drainage passage 28 forms a non-return valve 40 with a valve flap which is formed on only one side of the full plastic body 2 and accordingly deforms as a result of an acting negative pressure in order to allow exudate to pass through the drainage passage 28, but springs back to the initial position as shown in FIG. 8 when the suction pressure decreases. The walls laterally delimiting the drainage passage 28 are extended into the valve channel 38 and beyond the base 36, which further impedes the backflow of exudate into the drainage passage 28.

FIGS. 10 to 13 illustrate details of the wound treatment system with a hose connection element, which is characterized by reference sign 60. The hose connection element 60 is made of plastic and has a fastening base 62, which is flat on the lower side and is projected from the upper side by connectors 64, 66, which in the present case each form interfaces for connecting a hose. In the embodiment shown, it is assumed that the hose is a unitary hose having two lumens on the one hand for suction and on the other hand for rinsing. Thus, reference sign 64 characterizes the connector for the suction lumen of the hose and reference sign 66 characterizes the connector for the rinsing lumen of the hose. The hose itself is characterized by reference sign 68 in FIG. 14.

Obviously, the fastening base 62 almost completely projects beyond the sealing surface 16 and abuts against it in a sealed manner. The film 44 is located between the fastening base 62 and the surface of the full plastic body 2 and covers the collection channel 14, the discharge channel 18 as well as the rinsing channel 12 and the drainage channels 10 as well as the sealing surface 16 (cf. FIG. 12/13).

As FIG. 10 illustrates, the hose connection element 60 uses the hose connection web 26 to separate the suction side from the rinsing side. For this purpose, the hose connection element 60 forms an abutment web 70 between two bulges 72. Due to an elastic pretension, which is effected by abutting the fastening base 62 against the sealing surface 16 and fixing this abutment, the abutment web 70 lies against the free end of the hose connection web 26 in a fluid-tight manner.

FIG. 14 shows essential elements of a wound treatment by means of an embodiment of the wound treatment kit according to the invention. Reference sign 2 characterizes a full plastic body 2 which essentially covers a wound bed W and partially rests on wound margins R formed by the healthy skin surrounding the wound bed. Reference sign 44 characterizes the film which covers the full plastic body 2 on the upper side and is directly bonded thereto. Reference sign 80 characterizes a seal in the form of an incision film which circumferentially surrounds the full plastic body 2 and is bonded to the healthy skin. This incision film 80 seals the full plastic body 2 and the film 44 from the environment and fixes the full plastic body 2 to the body.

Reference sign 82 characterizes a wound dressing which is bonded to the lower side of the full plastic body 2 and is located between the wound bed W and the full plastic body 2. For this purpose, the full plastic body has on its rear side 5 an adhesive layer applied against the lower surface 6. This can be covered with a removable protective film before use.

Reference sign 60 characterizes the hose connection element, which protrudes through a slot 84 recessed in the film 44 and the seal 80 and via which the interior of the full plastic body 2 is connected to a hose 68 (cf. FIGS. 11 and 13). This hose 68 has a first lumen 86, which connects the hose connection element 60 to a collection container 88, which collects exudate that has been drawn off and is pressurized by a suction pump 90. Reference sign 92 characterizes a second lumen connecting the hose connection element 60 on the rinsing side to a rinsing valve 94. This rinsing valve 94 is controlled, in this case designed as a solenoid valve, and can be opened for cyclic rinsing. This introduces air from the environment on the front side 3 through the rinsing channel 12 into the drainage channels 10 of the full plastic body 2 and underneath the film 44. Drainage of exudate from the full plastic body 2 is improved and clogging in the suction line can be removed.

Obviously, the hose connection web 26 attaches the hose 68 for both suction and rinsing and is accordingly the suction and rinsing hose connection.

This type of wound treatment can per se be essential to the invention and may also indicate a method according to the invention for rinsing a wound dressing irrespective of the specific configuration of the same. It is primarily a matter of bringing air on the front side 3 under the cover 80 and—if present—under a further cover of the wound dressing—in the present case the film 44—in order to facilitate the removal of exudate.

The rinsing valve 94 is preferably connected in terms of control to the control system of the suction pump 90 and is usually set cyclically by the same.

FIG. 15 shows a variation on FIG. 14, in which the full plastic body 2 is shown in a sectional view. In contrast to the embodiment according to FIGS. 1, 1a, the full plastic body 2 has on its rear side 5, which is associated with the wound bed W, a plurality of instill channels 96 which extend parallel to the drainage channels 10 and which are closed towards the front side 3 and open towards the rear side 5. A perforated, i.e. liquid-permeable film 98 is applied to the front side 3 and bonded to the surface of the full plastic body 2.

A highly absorbent pad characterized by reference sign 100 is applied to this liquid-permeable film 98. This highly absorbent pad 100 comprises a non-woven fabric of highly absorbent filaments. This highly absorbent pad 100 stores exudate passing through the full plastic body 2. Thus, the seventh embodiment does not have a connection for a suction pump. However, a hose connection element or the like adapted to withdraw exudate by means of negative pressure may be provided to prolong the time for a change of the wound dressing before replacement by a fresh wound dressing.

Reference sign 102 characterizes an instill hose which communicates with the instill channels 96 of the full plastic body 2 via an instill connection 104. Instill fluid is supplied to the wound bed W via the instill hose 102. This instill fluid is not drawn off in the second variant, but remains on the wound bed W and has a therapeutic effect there.

In this embodiment, too, the full plastic body 2 together with the highly absorbent pad 100 is sealed from the environment by the seal 80 and bonded to the healthy skin. Between the wound bed W and the rear side 5 of the full plastic body 2 there is—as in the embodiment according to FIG. 14—a wound dressing 82 presently made of a foam.

In the embodiment shown in FIG. 16, the first embodiment of the full plastic body 2 is used, wherein instill fluid is also supplied, via an instill hose 102 inserted through the seal 80 into the wound dressing 82.

FIG. 16 further illustrates a variant to a hose connection element 60, which forms the two interfaces for the connectors 64 and 66 for the suction and the rinsing side.

In the embodiment according to FIG. 17, the full plastic body 2 is penetrated centrally by an instill passage 106, which forms an instill outlet 108 on the rear side 5, via which the instill fluid is discharged in the direction of the wound bed W, which communicates with an instill connection 104, which is presently formed on the circumferential edge of the full plastic body 2, which is formed on the hose connection element 60 as a third connector or as a third interface for the connection of a corresponding hose.

### LIST OF REFERENCE SIGNS

2 full plastic body
3 front side
4 upper surface
5 rear side
6 lower surface
8 outer edge
10 drainage channel
12 rinsing channel
14 collection channel
16 sealing surface
18 discharge channel
20 collecting trough
22 rinsing supply channel
24 rinsing trough
26 hose connection web
28 drainage passage
30 outlet opening
32 bottom
34 collar
36 bottom of the collar
38 valve channel
40 valve
42 channel walls
44 film
46 valve lug
48 valve opening
50 longitudinal slot
52 transverse valve channel
54 surface segment
60 hose connection element
62 fastening base
64 connector—suction side
66 connector—rinsing side
68 hose
70 abutment web
72 bulge
80 seal
82 wound dressing
84 slot
86 first lumen
88 collection container
90 suction pump
92 second lumen
94 rinsing valve
96 instill channel
98 liquid-permeable film
100 highly absorbent pad
102 instill hose
104 instill connection
108 instill outlet
106 instill passage
W wound bed
R wound margin

1. A wound treatment system comprising a seal adapted to be applied to a skin surrounding the wound and a fluid-permeable pad disposed between the seal and a wound bed (W) made of a flat, substantially planar full plastic body penetrated by drainage passages, at least one of the drainage passages having a valve.

2. The wound treatment system according to claim 1, in which the drainage passages communicate with drainage channels which are formed on a front side of the full plastic body to be provided facing away from the wound bed (W) and are closed to a rear side to be provided opposite thereto.

3. The wound treatment system according to claim 1, the valve forming an outlet opening which is exposed to a front side to be provided facing away from the wound bed (W) and which is surrounded by a valve channel which lies lower than the outlet opening.

4. The wound treatment system according to claim 3, an outlet opening which opens into the drainage channel being formed in a channel wall delimiting the drainage channel and which opens into the drainage channel below the surface and above a bottom of the drainage channel.

5. The wound treatment system according to claim 1, further comprising drainage channels delimited by channel walls and an outlet opening recessed in the channel wall.

6. The wound treatment system according to claim 5, the outlet opening being formed by valve lugs extending from the channel wall.

7. The wound treatment system according to claim 6, the valve lugs ending flush with a flat surface provided on the front side of the full plastic body.

8. The wound treatment system according to claim 1, and a hose connection formed in one piece on the full plastic body, which is arranged on the front side of the full plastic body and projects beyond an otherwise flat surface of the full plastic body.

9. The wound treatment system according to claim 8, and an enlarged sealing surface provided adjacent to the hose connection.

10. The wound treatment system according to claim 8, the hose connection being adapted for the connection of at least two lumens.

11. The wound treatment system according to claim 1, in which a hose connection element connectable to the full plastic body and positionable, in a sealing manner against the sealing surface, forms an interface for connecting a hose.

12. The wound treatment system according to claim 1, the full plastic body having a circumferential raised outer edge and the raised outer edge deliminting a rinsing channel which communicates with the drainage channels and a rinsing hose connection which communicates with a hose with which a rinsing valve is associated for the controlled inlet of a rinsing fluid.

13. The wound treatment system according to claim 1, further including an adhesive layer applied to the front side of the full plastic body and which connects a film to the full plastic body.

14. The wound treatment system according to claim 1, the drainage passage being delimited by valve lugs which cooperate with a film applied to the upper side of the full plastic body in such a way that, in the event of a suction pressure acting between the film and the full plastic body, the film is applied against the valve lugs and these are thereby spread to open the valve.

15. The wound treatment system according to claim 1, at least one instill outlet is provided on a rear side of the full plastic body to be provided facing the wound bed (W), preferably a plurality of instill channels open to the rear side are provided, and the full plastic body has an instill connection for an instill hose communicating with the instill outlet (108) or the instill channels.

16. The wound treatment system according to claim 1, in a sterile package comprising a full plastic body, and a film adhered to the full plastic body.

17. The wound treatment system according to claim 1, including a full plastic body.
